(19) United States
(12) Patent Application Publication
(10) Pub. No.: US 2026/0221219 A1
(43) Pub. Date: Jul. 30, 2026

(54) Method For Predicting Binding Structure Between Protein And Ligand

(71) Applicant: DEARGEN INC., Daejeon (KR)

(72) Inventors: Jimin Park, Seoul (KR); Daeseok LEE, Seoul (KR)

(73) Assignee: DEARGEN INC., Daejeon (KR)

(21) Appl. No.: 19/142,081

(22) PCT Filed: Oct. 24, 2023
(86) PCT No.: PCT/KR2023/016541

§ 371 Date: Sep. 29, 2025

(30) Foreign Application Priority Data
Dec. 21, 2022 (KR) 10-2022-0180016

(51) Int. Cl. G16B 15/30; G06N 3/045; G16B 20/30; G16B 40/20

(52) U.S. Cl. CPC G16B 15/30; G06N 3/045; G16B 20/30; G16B 40/20

(57) ABSTRACT

According to the present disclosure, a method for predicting a protein-ligand binding structure using a neural network model, performed by a computing device may include: obtaining a first pair representation related to a protein; obtaining a second pair representation related to a ligand; obtaining an interaction representation between the protein and the ligand; updating the first pair representation, the second pair representation, and the interaction representation; and predicting the protein-ligand binding structure after the updating, based on the first pair representation, the second pair representation, and the interaction representation.

### TECHNICAL FIELD

The present disclosure relates to a method for predicting a protein-ligand binding structure, and more particularly, to a method for predicting a protein-ligand binding structure by considering an interaction feature between the protein and the ligand.

### BACKGROUND ART

Today, designing or discovering a ligand that binds to a specific protein is one of the very important processes in developing drugs.

As conventional binding prediction methods for predicting the ligand that binds to the specific protein, methods for training a neural network model with structural or physical features of proteins and ligands to predict a protein-ligand binding structure have been widely used. However, the conventional binding prediction methods have primarily used methods that assume proteins and ligands are rigid bodies that do not change upon binding in order to reduce model complexity. In other words, the conventional binding prediction methods have mainly employed docking schemes (e.g., a rigid docking scheme) that do not consider structural changes caused by protein and ligand binding. That is, the conventional methods have problems such as “inability to specify protein binding sites” and “inability to predict structural changes of proteins and ligands”.

Therefore, there is a need for a method for predicting a protein-ligand binding structure that can address the problems.

On the other hand, the present disclosure has been derived at least based on the technical background described above, but the technical problem or object of the present disclosure is not limited to solving the problems or disadvantages described above. That is, the present disclosure may cover various technical issues related to the content to be described below, in addition to the technical issues discussed above.

### DISCLOSURE

### Technical Problem

The present disclosure has been made in an effort to improve a performance of predicting a protein-ligand binding structure.

Meanwhile, a technical object to be achieved by the present disclosure is not limited to the above-mentioned technical object, and various technical objects can be included within the scope which is obvious to those skilled in the art from contents to be described below.

### Technical Solution

In order to achieve the object, according to an embodiment of the present disclosure, a method for predicting a protein-ligand binding structure using a neural network model, performed by at least one computing device is disclosed. Here, the method may include: obtaining a first pair representation related to a protein; obtaining a second pair representation related to a ligand; obtaining an interaction representation between the protein and the ligand; updating the first pair representation, the second pair representation, and the interaction representation; and predicting the protein-ligand binding structure after the updating, based on the first pair representation, the second pair representation, and the interaction representation.

As an embodiment, the obtaining of the first pair representation related to the protein may include: calculating a one-dimensional feature vector based on one-dimensional information related to a structure or a property of the protein; calculating a two-dimensional feature vector based on two-dimensional information related to the structure or the property of the protein; and obtaining the first pair representation based on the one-dimensional feature vector and the two-dimensional feature vector.

As an embodiment, the one-dimensional feature vector may include a feature related to an individual amino acid residue of the protein, and the two-dimensional feature vector may include features related to two amino acid residues of the protein.

As an embodiment, the obtaining of the first pair representation may include obtaining pair representations related to an i-th amino acid residue of the protein and a j-th amino acid residue of the protein based on a one-dimensional feature vector for the i-th amino acid residue, a one-dimensional feature vector for the j-th amino acid residue of, and a two-dimensional feature vector for the i-th amino acid residue and the j-th amino acid residue, and the i and j may be natural numbers.

As an embodiment, the obtaining of the second pair representation related to the ligand may include: calculating a one-dimensional feature vector based on a one-dimensional information related to a structure or a property of the ligand; calculating a two-dimensional feature vector based on a two-dimensional information related to the structure or the property of the ligand; and obtaining the second pair representation based on the one-dimensional feature vector and the two-dimensional feature vector.

As an embodiment, the one-dimensional feature vector may include a feature related to an individual atom of the ligand, and the two-dimensional feature vector may include features related to two atoms of the ligand.

As an embodiment, the obtaining of the second pair representation may include obtaining a pair representations related to an i-th atom of the ligand and a j-th atom of the ligand based on a one-dimensional feature vector for the i-th atom, a one-dimensional feature vector for the j-th atom, and a two-dimensional feature vector for the i-th atom and the j-th atom, and the i and j may be natural numbers.

As an embodiment, the obtaining of the interaction representation between the protein and the ligand may include obtaining the interaction representation between the protein and the ligand by using a one-dimensional feature vector calculated based on one-dimensional information of the protein and a one-dimensional feature vector calculated based on one-dimensional information of the ligand.

As an embodiment, the obtaining of the interaction representation between the protein and the ligand may include obtaining an interaction representation related to an i-th atom of the ligand and a j-th amino acid residue of the protein by using a one-dimensional feature vector for the i-th atom of the ligand and a one-dimensional feature vector for the j-th amino acid residue of the protein, and the i and j may be natural numbers. As an embodiment, the updating may include updating the first pair representation, the second pair representation, and the interaction representation by using a first neural network model, and the first neural network model may include: a first-first neural network model that independently updates each of the first pair representation, the second pair representation, and the interaction representation; a first-second neural network model that updates at least one of the first pair representation or the second pair representation by using the interaction representation; and a first-third neural network model that updates the interaction representation by using at least one of the first pair representation or the second pair representation.

As an embodiment, the first-first neural network model may include a neural network model that independently updates each of the first pair representation, the second pair representation, and the interaction representation by using a self-attention mechanism. As an embodiment, the first-second neural network model may include at least one of a neural network model that updates a pair representation associated with a n i-th amino acid residue of the protein and a j-th amino acid residue of the protein, based on an interaction representation associated with the i-th amino acid residue of the protein and an interaction representation associated with a j-th amino acid residue of the protein; or a neural network model that updates a pair representation associated with an i-th atom of the ligand and a j-th atom of the ligand, based on an interaction representation associated with the i-th atom of the ligand and an interaction representation associated with the j-th atom of the ligand, and the i and j may be natural numbers.

As an embodiment, the first-third neural network model may include at least one of a neural network model that updates an interaction representation related to an i-th atom of the ligand by using cross-attention mechanism that refers to the first pair representation related to the protein; or a neural network model that updates an interaction representation related to a j-th amino acid residue of the protein by using a cross-attention mechanism that refers to the second pair representation related to the ligand, and the i and j may be natural numbers.

As an embodiment, the updating may include at least one of updating a first matrix including a plurality of first pair representations related to the protein; or updating a second matrix including a plurality of second pair representations related to the ligand, and at least one of the first matrix or the second matrix may be updated to become a symmetric matrix.

As an embodiment, the predicting of the binding structure between the protein and the ligand may include predicting the binding structure between the protein and the ligand by using a second neural network model, and the second neural network model may include: a second-first neural network model that calculates a protein representation by using the first pair representation and the interaction representation; a second-second neural network model that calculates a ligand representation by using the second pair representation and the interaction representation; and a second-third neural network model that calculates the binding structure between the protein and the ligand by using the protein representation, the ligand representation, and the interaction representation.

As an embodiment, the second-third neural network model may include a neural network model that utilizes the interaction representation when applying a cross-attention mechanism between the protein representation and the ligand representation.

As an embodiment, the predicting of the binding structure between the protein and the ligand may include: calculating distance information related to the protein or the ligand based on the first pair representation, the second pair representation, and the interaction representation by using a third neural network model; and predicting the binding structure between the protein and the ligand by using a first scoring function based on the distance information and a second scoring function based on physics.

As an embodiment, the method may further include: re-obtaining the first pair representation, the second pair representation, and the interaction representation based on the predicted binding structure; updating the first pair representation, the second pair representation, and the interaction representation; and re-predicting the binding structure between the protein and the ligand after the updating, based on the first pair representation, the second pair representation, and the interaction representation.

As an embodiment, the method may further include calculating a loss function for training the neural network model, and the calculating of the loss function may further include calculating an auxiliary loss function based on the interaction information between the protein and the ligand.

Further, in order to achieve the object, a device according to an embodiment of the present disclosure is disclosed. Here, the device may include: at least one processor; and a memory, and the processor may be configured to obtain a first pair representation related to a protein; obtain a second pair representation related to a ligand; obtain an interaction representation between the protein and the ligand; update the first pair representation, the second pair representation, and the interaction representation; and predict a binding structure between the protein and the ligand after the updating, based on the first pair representation, the second pair representation, and the interaction representation.

Further, in order to achieve the object, a computer program stored in a computer-readable storage medium according to an embodiment of the present disclosure is disclosed. Herein, when the program is executed by at least one processor, the program may allow the at least one processor to perform operations of predicting a binding structure between a protein and a ligand, and the operations may include: an operation of obtaining a first pair representation related to a protein; an operation of obtaining a second pair representation related to a ligand; an operation of obtaining an interaction representation between the protein and the ligand; an operation of updating the first pair representation, the second pair representation, and the interaction representation; and an operation of predicting the binding structure between the protein and the ligand after the updating, based on the first pair representation, the second pair representation, and the interaction representation.

### Advantageous Effects

According to the present disclosure, it is possible to improve a performance of predicting a protein-ligand binding structure. For example, according to the present disclosure, it is possible to predict a binding structure in which a ligand binding site within the protein and a structural change due to binding are reflected, when predicting the protein-ligand binding structure. Furthermore, according to the present disclosure, it is possible to more accurately predict the binding structure of the protein and the ligand by considering a predicted interaction feature between the protein and the ligand together when predicting the protein-ligand binding structure.

Meanwhile, the effects of the present disclosure are not limited to the above-mentioned effects, and various effects can be included within the scope which is obvious to those skilled in the art from contents to be described below.

### DESCRIPTION OF DRAWINGS

FIG. 1 is a block diagram of a computing device performing operations according to an embodiment of the present disclosure.

FIG. 2 is a schematic diagram illustrating a neural network model according to an embodiment of the present disclosure.

FIG. 3 is a flowchart illustrating a general method for predicting a protein-ligand binding structure according to an embodiment of the present disclosure.

FIG. 4 is a schematic diagram illustrating a method for predicting a protein-ligand binding structure according to an embodiment of the present disclosure.

FIG. 5 is a schematic diagram specifically illustrating a method for predicting a protein-ligand binding structure according to an embodiment of the present disclosure.

FIG. 6 is a schematic diagram illustrating a specific method for predicting the protein-ligand binding structure by using the neural network model according to an embodiment of the present disclosure.

FIG. 7 is a schematic diagram illustrating an example of a method for updating a protein or ligand representation according to an embodiment of the present disclosure.

FIG. 8 is a simple and normal schematic diagram of an exemplary computing environment in which the embodiments of the present disclosure may be implemented.

### BEST MODE

Various exemplary embodiments will now be described with reference to drawings. In the present specification, various descriptions are presented to provide appreciation of the present disclosure. However, it is apparent that the exemplary embodiments can be executed without the specific description.

“Component”, “module”, “system”, and the like which are terms used in the specification refer to a computer-related entity, hardware, firmware, software, and a combination of the software and the hardware, or execution of the software. For example, the component may be a processing procedure executed on a processor, the processor, an object, an execution thread, a program, and/or a computer, but is not limited thereto. For example, both an application executed in a computing device and the computing device may be the components. One or more components may reside within the processor and/or a thread of execution. One component may be localized in one computer. One component may be distributed between two or more computers. Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

The term “or” is intended to mean not exclusive “or” but inclusive “or”. That is, when not separately specified or not clear in terms of a context, a sentence “X uses A or B” is intended to mean one of the natural inclusive substitutions. That is, the sentence “X uses A or B” may be applied to any of the case where X uses A, the case where X uses B, or the case where X uses both A and B. Further, it should be understood that the term “and/or” used in this specification designates and includes all available combinations of one or more items among enumerated related items.

It should be appreciated that the term “comprise” and/or “comprising” means presence of corresponding features and/or components. However, it should be appreciated that the term “comprises” and/or “comprising” means that presence or addition of one or more other features, components, and/or a group thereof is not excluded. Further, when not separately specified or it is not clear in terms of the context that a singular form is indicated, it should be construed that the singular form generally means “one or more” in this specification and the claims.

The term “at least one of A or B” should be interpreted to mean “a case including only A”, “a case including only B”, and “a case in which A and B are combined”.

Those skilled in the art need to recognize that various illustrative logical blocks, configurations, modules, circuits, means, logic, and algorithm steps described in connection with the exemplary embodiments disclosed herein may be additionally implemented as electronic hardware, computer software, or combinations of both sides. To clearly illustrate the interchangeability of hardware and software, various illustrative components, blocks, configurations, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented exemplary embodiments is provided so that those skilled in the art of the present disclosure use or implement the present disclosure. Various modifications to the exemplary embodiments will be apparent to those skilled in the art. Generic principles defined herein may be applied to other embodiments without departing from the scope of the present disclosure. Therefore, the present disclosure is not limited to the exemplary embodiments presented herein. The present disclosure should be analyzed within the widest range which is coherent with the principles and new features presented herein.

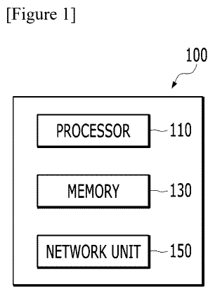

A configuration of the computing device 100 illustrated in FIG. 1 is only an example shown through simplification. In an exemplary embodiment of the present disclosure, the computing device 100 may include other components for performing a computing environment of the computing device 100 and only some of the disclosed components may constitute the computing device 100.

The computing device 100 may include a processor 110, a memory 130, and a network unit 150.

The processor 110 may be constituted by one or more cores and may include processors for data analysis and deep learning, which include a central processing unit (CPU), a general purpose graphics processing unit (GPGPU), a tensor processing unit (TPU), and the like of the computing device. The processor 110 may read a computer program stored in the memory 130 to perform data processing for machine learning according to an exemplary embodiment of the present disclosure. According to an exemplary embodiment of the present disclosure, the processor 110 may perform a calculation for learning the neural network. The processor 110 may perform calculations for learning the neural network, which include processing of input data for learning in deep learning (DL), extracting a feature in the input data, calculating an error, updating a weight of the neural network using backpropagation, and the like. At least one of the CPU, GPGPU, and TPU of the processor 110 may process learning of a network function. For example, both the CPU and the GPGPU may process the learning of the network function and data classification using the network function. Further, in an exemplary embodiment of the present disclosure, processors of a plurality of computing devices may be used together to process the learning of the network function and the data classification using the network function. Further, the computer program executed in the computing device according to an exemplary embodiment of the present disclosure may be a CPU, GPGPU, or TPU executable program.

According to an embodiment of the present disclosure, the memory (130) may store information of any form generated or determined by the processor (110), as well as information of any form received by the network unit (150).

According to an embodiment of the present disclosure, the memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The above description of the memory is just an example and the present disclosure is not limited thereto.

The network unit 150 according to an embodiment of the present disclosure may use various wired communication systems such as public switched telephone network (PSTN), x digital subscriber line (xDSL), rate adaptive DSL (RADSL), multi rate DSL (MDSL), very high speed DSL (VDSL), universal asymmetric DSL (UADSL), high bit rate DSL (HDSL), and local area network (LAN).

Further, the network unit 150 presented in the present disclosure may use various wireless communication systems such as code division multi access (CDMA), time division multi access (TDMA), frequency division multi access (FDMA), orthogonal frequency division multi access (OFDMA), single carrier-FDMA (SC-FDMA), and other systems.

In the present disclosure, the network unit 150 may be configured regardless of communication modes such as wired and wireless modes and constituted by various communication networks including a personal area network (PAN), a wide area network (WAN), and the like. Further, the network may be known World Wide Web (WWW) and may adopt a wireless transmission technology used for short-distance communication, such as infrared data association (IrDA) or Bluetooth. The techniques described in the present disclosure may also be used in other networks mentioned above.

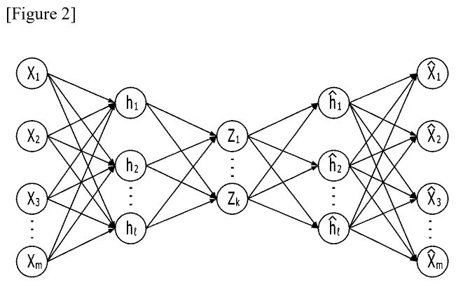

FIG. 2 is a schematic diagram illustrating a neural network model according to an embodiment of the present disclosure.

Throughout the present disclosure, the terms “neural network model” and “neural network” may be used interchangeably. A neural network model may generally be composed of a set of interconnected computational units, which may be referred to as nodes. These nodes may also be referred to as neurons. The neural network model includes at least one node. The nodes (or neurons) constituting the neural network model may be interconnected via one or more links.

In the neural network, one or more nodes connected through the link may relatively form the relationship between an input node and an output node. Concepts of the input node and the output node are relative and a predetermined node which has the output node relationship with respect to one node may have the input node relationship in the relationship with another node and vice versa. As described above, the relationship of the input node to the output node may be generated based on the link. One or more output nodes may be connected to one input node through the link and vice versa.

In the relationship of the input node and the output node connected through one link, a value of data of the output node may be determined based on data input in the input node. Here, a link connecting the input node and the output node to each other may have a weight. The weight may be variable and may vary by a user or an algorithm in order for the neural network to perform a desired function. For example, when one or more input nodes are mutually connected to one output node by the respective links, the output node may determine an output node value based on values input in the input nodes connected with the output node and the weights set in the links corresponding to the respective input nodes.

As described above, in the neural network, one or more nodes are connected to each other through one or more links to form a relationship of the input node and output node in the neural network. A characteristic of the neural network may be determined according to the number of nodes, the number of links, correlations between the nodes and the links, and values of the weights, granted to the respective links, in the neural network. For example, when the same number of nodes and links exist and there are two neural networks in which the weight values of the links are different from each other, it may be recognized that two neural networks are different from each other.

The neural network may be constituted by a set of one or more nodes. A subset of the nodes constituting the neural network may constitute a layer. Some of the nodes constituting the neural network may constitute one layer based on the distances from the initial input node. For example, a set of nodes of which distance from the initial input node is n may constitute n layers. The distance from the initial input node may be defined by the minimum number of links which should be passed through for reaching the corresponding node from the initial input node. However, definition of the layer is predetermined for description and the order of the layer in the neural network may be defined by a method different from the aforementioned method. For example, the layers of the nodes may be defined by the distance from a final output node.

The initial input node may mean one or more nodes in which data is directly input without passing through the links in the relationships with other nodes among the nodes in the neural network. Alternatively, in the neural network, in the relationship between the nodes based on the link, the initial input node may mean nodes which do not have other input nodes connected through the links. Similarly thereto, the final output node may mean one or more nodes which do not have the output node in the relationship with other nodes among the nodes in the neural network. Further, a hidden node may mean nodes constituting the neural network other than the initial input node and the final output node.

The neural network which may be used in the artificial intelligence based model of the present disclosure may be trained in at least one scheme of supervised learning, unsupervised learning, semi supervised learning, transfer learning, active learning, or reinforcement learning. The training of the neural network may be a process in which the neural network applies knowledge for performing a specific operation to the neural network.

The neural network may be trained in a direction to minimize errors of an output. The training of the neural network is a process of repeatedly inputting learning data into the neural network and calculating the output of the neural network for the learning data and the error of a target and back-propagating the errors of the neural network from the output layer of the neural network toward the input layer in a direction to reduce the errors to update the weight of each node of the neural network. In the case of the supervised learning, the learning data in which each training data is labeled with a correct answer (i.e., the labeled learning data) may be used, and in the case of the unsupervised learning each training data may not be labeled with a correct answer. That is, for example, the learning data in the case of the supervised learning associated with the data classification may be data in which each training data is labeled with a category. The labeled learning data is input to the neural network, and the error may be calculated by comparing the output (category) of the neural network with the label of the learning data. As another example, in the case of the unsupervised learning associated with the data classification, the learning data as the input may be compared with the output of the neural network to calculate the error. The calculated error may be back-propagated in a reverse direction (i.e., a direction from the output layer toward the input layer) in the neural network, and connection weights of respective nodes of each layer of the neural network may be updated according to the back propagation. A variation amount of the updated connection weight of each node may be determined according to a learning rate. Calculation of the neural network for the input data and the back-propagation of the error may constitute a learning cycle (epoch). The learning rate may be applied differently according to the number of repetition times of the learning cycle of the neural network. For example, in an initial stage of the learning of the neural network, the neural network may ensure a certain level of performance quickly by using a high learning rate, thereby increasing efficiency, and may use a low learning rate in a latter stage of the learning, thereby increasing accuracy.

In the training of the neural network, the learning data may be generally a subset of actual data (i.e., data to be processed using the trained neural network), and as a result, there may exist a learning cycle in which errors for the learning data decrease, but the errors for the actual data increase. Overfitting is a phenomenon in which the errors for the actual data increase due to excessive learning of the learning data. For example, a phenomenon in which the neural network that learns a cat while seeing a yellow cat does not recognize a cat other than a yellow cat as a cat may be an example of overfitting. The overfitting may act as a cause which increases the error of the machine learning algorithm. Various optimization methods may be used in order to prevent the overfitting. In order to prevent the overfitting, a method such as increasing the learning data, regularization, dropout of deactivating a part of the node of the network in the process of learning, utilization of a batch normalization layer, etc., may be applied.

Disclosed is a computer readable medium storing the data structure according to an exemplary embodiment of the present disclosure.

The data structure may refer to organization, management, and storage of data that enable efficient access and modification of data. The data structure may refer to organization of data for solving a specific problem (for example, data search, data storage, and data modification in the shortest time). The data structure may also be defined with a physical or logical relationship between the data elements designed to support a specific data processing function. A logical relationship between data elements may include a connection relationship between user defined data elements. A physical relationship between data elements may include an actual relationship between the data elements physically stored in a computer readable storage medium (for example, a permanent storage device). In particular, the data structure may include a set of data, a relationship between data, and a function or a command applicable to data. Through the effectively designed data structure, the computing device may perform a calculation while minimally using resources of the computing device. In particular, the computing device may improve efficiency of calculation, reading, insertion, deletion, comparison, exchange, and search through the effectively designed data structure.

The data structure may be divided into a linear data structure and a non-linear data structure according to the form of the data structure. The linear data structure may be the structure in which only one data is connected after one data. The linear data structure may include a list, a stack, a queue, and a deque. The list may mean a series of dataset in which order exists internally. The list may include a linked list. The linked list may have a data structure in which data is connected in a method in which each data has a pointer and is linked in a single line. In the linked list, the pointer may include information about the connection with the next or previous data. The linked list may be expressed as a single linked list, a double linked list, and a circular linked list according to the form. The stack may have a data listing structure with limited access to data. The stack may have a linear data structure that may process (for example, insert or delete) data only at one end of the data structure. The data stored in the stack may have a data structure (Last In First Out, LIFO) in which the later the data enters, the sooner the data comes out. The queue is a data listing structure with limited access to data, and may have a data structure (First In First Out, FIFO) in which the later the data is stored, the later the data comes out, unlike the stack. The deque may have a data structure that may process data at both ends of the data structure.

The non-linear data structure may be the structure in which the plurality of data is connected after one data. The non-linear data structure may include a graph data structure. The graph data structure may be defined with a vertex and an edge, and the edge may include a line connecting two different vertexes. The graph data structure may include a tree data structure. The tree data structure may be the data structure in which a path connecting two different vertexes among the plurality of vertexes included in the tree is one. That is, the tree data structure may be the data structure in which a loop is not formed in the graph data structure.

The data structure may include a neural network. Further, the data structure including the neural network may be stored in a computer readable medium. The data structure including the neural network may also include preprocessed data for processing by the neural network, data input to the neural network, a weight of the neural network, a hyper-parameter of the neural network, data obtained from the neural network, an active function associated with each node or layer of the neural network, and a loss function for training of the neural network. The data structure including the neural network may include predetermined configuration elements among the disclosed configurations. That is, the data structure including the neural network may include the entirety or a predetermined combination of pre-processed data for processing by neural network, data input to the neural network, a weight of the neural network, a hyper parameter of the neural network, data obtained from the neural network, an active function associated with each node or layer of the neural network, and a loss function for training the neural network. In addition to the foregoing configurations, the data structure including the neural network may include predetermined other information determining a characteristic of the neural network. Further, the data structure may include all type of data used or generated in a computation process of the neural network, and is not limited to the foregoing matter. The computer readable medium may include a computer readable recording medium and/or a computer readable transmission medium. The neural network may be formed of a set of interconnected calculation units which are generally referred to as “nodes”. The “nodes” may also be called “neurons.” The neural network consists of one or more nodes.

The data structure may include data input to the neural network. The data structure including the data input to the neural network may be stored in the computer readable medium. The data input to the neural network may include training data input in the training process of the neural network and/or input data input to the training completed neural network. The data input to the neural network may include data that has undergone pre-processing and/or data to be pre-processed. The pre-processing may include a data processing process for inputting data to the neural network. Accordingly, the data structure may include data to be pre-processed and data generated by the pre-processing. The foregoing data structure is merely an example, and the present disclosure is not limited thereto.

The data structure may include a weight of the neural network (in the present specification, weights and parameters may be used with the same meaning), Further, the data structure including the weight of the neural network may be stored in the computer readable medium. The neural network may include a plurality of weights. The weight is variable, and in order for the neural network to perform a desired function, the weight may be varied by a user or an algorithm. For example, when one or more input nodes are connected to one output node by links, respectively, the output node may determine a data value output from the output node based on values input to the input nodes connected to the output node and the weight set in the link corresponding to each of the input nodes. The foregoing data structure is merely an example, and the present disclosure is not limited thereto.

For a non-limited example, the weight may include a weight varied in the neural network training process and/or the weight when the training of the neural network is completed. The weight varied in the neural network training process may include a weight at a time at which a training cycle starts and/or a weight varied during a training cycle. The weight when the training of the neural network is completed may include a weight of the neural network completing the training cycle. Accordingly, the data structure including the weight of the neural network may include the data structure including the weight varied in the neural network training process and/or the weight when the training of the neural network is completed. Accordingly, it is assumed that the weight and/or a combination of the respective weights are included in the data structure including the weight of the neural network. The foregoing data structure is merely an example, and the present disclosure is not limited thereto.

The data structure including the weight of the neural network may be stored in the computer readable storage medium (for example, a memory and a hard disk) after undergoing a serialization process. The serialization may be the process of storing the data structure in the same or different computing devices and converting the data structure into a form that may be reconstructed and used later. The computing device may serialize the data structure and transceive the data through a network. The serialized data structure including the weight of the neural network may be reconstructed in the same or different computing devices through deserialization. The data structure including the weight of the neural network is not limited to the serialization. Further, the data structure including the weight of the neural network may include a data structure (for example, in the non-linear data structure, B-Tree, Trie, m-way search tree, AVL tree, and Red-Black Tree) for improving efficiency of the calculation while minimally using the resources of the computing device. The foregoing matter is merely an example, and the present disclosure is not limited thereto.

The data structure may include a hyper-parameter of the neural network. The data structure including the hyper-parameter of the neural network may be stored in the computer readable medium. The hyper-parameter may be a variable varied by a user. The hyper-parameter may include, for example, a learning rate, a cost function, the number of times of repetition of the training cycle, weight initialization (for example, setting of a range of a weight value to be weight-initialized), and the number of hidden units (for example, the number of hidden layers and the number of nodes of the hidden layer). The foregoing data structure is merely an example, and the present disclosure is not limited thereto.

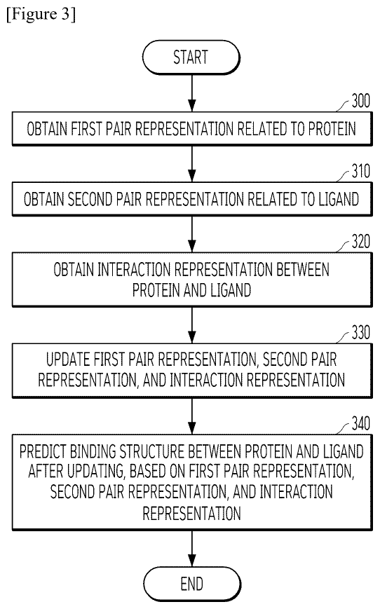

A general process for the method for predicting the binding structure between the protein and ligand will now be described with reference to FIG. 3.

Referring to FIG. 3, a processor 110 may perform steps S300 to S340 in order to predict a protein-ligand binding structure. In this case, steps S300 to S340 above may include, by the processor 110, “a step of obtaining a first pair representation related to the protein in order to predict the binding structure” (S300), “a step of obtaining a second pair representation related to the ligand” (S310), “a step of obtaining an interaction representation between the protein and the ligand” (S320), “a step of updating the first pair representation, the second pair representation, and the interaction representation” (S330), and “a step of predicting the protein-ligand binding structure after the updating, based on the first pair representation, the second pair representation, and the interaction representation” (S340).

Step S300 above is a step of obtaining the first pair representation related to the protein in order to predict the binding structure.

In relation to step S300 above, the processor 110 may perform steps including “a step of calculating a one-dimensional feature vector based on one-dimensional information related to a structure or a property of the protein” (S301), “a step of calculating a two-dimensional feature vector based on two-dimensional information related to the structure or the property of the protein” (S302), and “a step of obtaining the first pair representation based on the one-dimensional feature vector and the two-dimensional feature vector” (S303).

In relation to step S301 above, the one-dimensional information may include feature (e.g., a structural feature, a physical/chemical property feature, etc.) information related to individual amino acid residues of the protein for which binding structure is to be predicted. For example, the one-dimensional information may include types of individual amino acids, indices of individual amino acids, a torsion angle of a backbone for each individual amino acid residue, a hidden layer activation value of a neural network model (e.g., a neural network model such as AlphaFold that receives an amino acid sequences as an input), various physical/chemical property information related to individual amino acids, etc. Meanwhile, the indices of the individual amino acids may be determined based on residue numbers of individual amino acid residues, but are not limited thereto, and may be determined by other criteria or in an arbitrary order. Furthermore, the one-dimensional information may include other types of information that may be represented one-dimensionally in relation to the protein for which binding structure is to be predicted. The one-dimensional information may be obtained from sources including: sequence information of the protein for which binding structure is to be predicted, known structure information of the protein (e.g., coordinate information extracted from databases), known property information of the protein (e.g., physical/chemical property information extracted from databases), structure information predicted by other models in relation to the protein (e.g., structure information predicted by models such as ‘Deep Dock’, ‘EquiBind’, ‘AlphaFold’, etc.), structure information given as initial input values of the model, structure information predicted in previous prediction steps in relation to the protein (e.g., when structure prediction operations are repeated, the structure information predicted in the previous prediction steps is re-input as initial values, so the one-dimensional information may be re-extracted based on the structure information predicted in the previous prediction steps), etc. Meanwhile, since the one-dimensional feature vector is calculated based on such one-dimensional information, the one-dimensional feature vector may include features related to individual amino acid residues of the protein for which binding structure is to be predicted. In an embodiment, the one-dimensional feature vector may be generated based on concatenating all or some of the features included in the one-dimensional information in vector dimensions. Furthermore, the one-dimensional feature vector may be generated for each of the amino acid residues included in the protein. For example, a one-dimensional feature vector for an i-th amino acid residue of the protein may be represented as PFᵢ, etc.

In relation to step S302 above, the two-dimensional information may include feature (e.g., a structural feature, a physical/chemical property feature, etc.) information related to two amino acid residues of the protein for which binding structure is to be predicted. For example, the two-dimensional information may include a relationship index between two amino acid residues (e.g., when indices of two amino acid residues are i and j, the relationship index may be determined as i-j, etc.), a distance between two amino acid residues, an angle between two amino acid residues, a hidden layer activation value of a neural network model (e.g., a neural network model such as AlphaFold that receives an amino acid sequence as an input), various physical/chemical property information related to two amino acid residues, etc. Furthermore, the two-dimensional information may include other types of information that may be represented two-dimensionally in relation to the protein for which binding structure is to be predicted. The two-dimensional information may be obtained from sources including: sequence information of the protein for which binding structure is to be predicted, known structure information of the protein (e.g., coordinate information extracted from databases), known property information of the protein (e.g., physical/chemical property information extracted from databases), structure information predicted by other models in relation to the protein (e.g., structure information predicted by models such as ‘Deep Dock’, ‘EquiBind’, ‘AlphaFold’, etc.), structure information given as initial input values of the model, structure information predicted in previous prediction steps in relation to the protein (e.g., when structure prediction operations are repeated, the structure information predicted in the previous prediction steps is re-input as initial values, so the two-dimensional information may be re-extracted based on the structure information predicted in the previous prediction steps), etc. Meanwhile, since the two-dimensional feature vector is calculated based on such two-dimensional information, the two-dimensional feature vector may include features related to two amino acid residues of the protein for which binding structure is to be predicted. In an embodiment, the two-dimensional feature vector may be generated based on concatenating all or some of the features included in the two-dimensional information in vector dimensions. Furthermore, the two-dimensional feature vector may be generated for each of all amino pairs, which may be generated by using amino acids included in the protein (in other words, when the protein includes n amino acid residues, n×n two-dimensional feature vectors may be generated. For example, a two-dimensional feature vector for an i-th amino acid residue of the protein, and the j-th amino acid residue may be represented as PFᵢⱼ, etc.

In relation to step S303 above, the processor 110 may obtain a first pair representation associated with the protein based on the one-dimensional feature vector and the two-dimensional feature vector. For example, the processor 110 may obtain a pair representation related to the i-th amino acid residue and the j-th amino acid residue based on “the one-dimensional feature vector for the i-th amino acid residue of the protein”, “the one-dimensional feature vector for the j-th amino acid residue of the protein”, and “the two-dimensional feature vector for the i-th amino acid residue and the j-th amino acid residue”. In an embodiment, PFᵢⱼ which a pair representation related to the i-th amino acid residue and the j-th amino acid residue may correspond to Linear(concat(PFᵢⱼ, PFᵢ, PFⱼ) when the one-dimensional feature vector for the i-th amino acid residue of the protein is PFᵢ, the one-dimensional feature vector for the j-th amino acid residue of the protein is PFⱼ, and the two-dimensional feature vector for the i-th amino acid residue and the j-th amino acid residue is PFᵢⱼ. In other words, PFᵢⱼ may be embedded based on concatenating PFᵢⱼ, PFᵢ, PFⱼ, and applying a linear function to concatenated vectors. Meanwhile, the first pair representation may be generated for each of all amino pairs, which may be generated by using the amino acid residues included in the protein (in other words, when the protein includes n amino acid residues, n×n first pair representations may be generated).

Step S310 above is a step of obtaining a second pair representation related to a ligand. In relation to step S310 above, the processor 110 may perform steps including “a step of calculating a one-dimensional feature vector based on one-dimensional information related to a structure or a property of the ligand” (S311), “a step of calculating a two-dimensional feature vector based on two-dimensional information related to the structure or the property of the ligand” (S312), and “a step of obtaining the second pair representation based on the one-dimensional feature vector and the two-dimensional feature vector” (S313).

In relation to step S301 above, the one-dimensional information may include feature (e.g., a structural feature, a physical/chemical property feature, etc.) information related to individual atoms of the ligand. For example, the one-dimensional information may include types of individual atoms, indices of individual atoms, aromaticity information of individual atoms, charge information of individual atoms, various physical/chemical property information related to individual atoms, etc. Furthermore, the one-dimensional information may include other types of information that may be represented one-dimensionally in relation to the ligand. Meanwhile, the indices of the individual atoms may be determined based on which character string individual atoms correspond to in a Simplified molecular-input line-entry system (SMILES), but are not limited thereto, and may be determined by other criteria or in an arbitrary order. The one-dimensional information may be obtained from sources including: atom-bond graphs with aromaticity and charge indicated (which may be given as character string representations such as SMILES, SELFIES, etc.), known structure information of the ligand (e.g., coordinate information extracted from a database), known property information of the ligand (e.g., physical/chemical property information extracted from a database), structure information predicted by other models in relation to the ligand, structure information of the ligand given as an initial input value of the model, structure information predicted in relation to the ligand in a previous prediction step (e.g., when a structure prediction operation is repeated, the structure information predicted in the previous prediction step is re-input as the initial value, so that the one-dimensional information may be re-extracted based on the structure information predicted in the previous prediction step), etc. Meanwhile, since the one-dimensional feature vector is calculated based on such one-dimensional information, the one-dimensional feature vector may include features related to individual atoms of the ligand. In an embodiment, the one-dimensional feature vector may be generated based on concatenating all or some of the features included in the one-dimensional information in vector dimensions. Furthermore, the one-dimensional feature vector may be generated for each of the atoms included in the ligand. For example, a one-dimensional feature vector for an i-th atom of the ligand may be represented as LFᵢ, etc.

In relation to step S312 above, the two-dimensional information may include feature (e.g., a structural feature, a physical/chemical property feature, etc.) information related to two atoms of the ligand. For example, the two-dimensional information may include a relationship index between two atoms (e.g., when indices of two atoms are i and j, the relationship index may be determined as i-j, etc.), a distance between two atoms, a bond order between two atoms (single bond, double bond, triple bond, etc.), various physical/chemical property information related to two atoms, etc. Furthermore, the two-dimensional information may include other types of information that may be represented two-dimensionally in relation to the ligand. The two-dimensional information may be obtained from sources including: atom-bond graphs with aromaticity and charge indicated (which may be given as character string representations such as SMILES, SELFIES, etc.), known structure information of the ligand (e.g., coordinate information extracted from a database), known property information of the ligand (e.g., physical/chemical property information extracted from a database), structure information predicted by other models in relation to the ligand, structure information of the ligand given as an initial input value of the model, structure information predicted in relation to the ligand in a previous prediction step (e.g., when a structure prediction operation is repeated, the structure information predicted in the previous prediction step is re-input as the initial value, so that the two-dimensional information may be re-extracted based on the structure information predicted in the previous prediction step), etc. Meanwhile, since the two-dimensional feature vector is calculated based on such two-dimensional information, the two-dimensional feature vector may include features related to two atoms of the ligand. In an embodiment, the two-dimensional feature vector may be generated based on concatenating all or some of the features included in the two-dimensional information in vector dimensions. Furthermore, the two-dimensional feature vector may be generated for each of all atoms pairs, which may be generated by using the atoms included in the protein (in other words, when the ligand includes m atoms, n×n two-dimensional feature vectors may be generated). For example, a two-dimensional feature vector for an i-th atom of the ligand and the j-th atom may be represented as LFᵢⱼ, etc.

In relation to step S313 above, the processor 110 may obtain a second pair representation associated with the ligand based on the one-dimensional feature vector and the two-dimensional feature vector. For example, the processor 110 may obtain a pair representation related to the i-th atom and the j-th atom based on “a one-dimensional feature vector for the i-th atom of the ligand”, “a one-dimensional feature vector for the j-th atom of the ligand”, and “a two-dimensional feature vector for the i-th atom and the j-th atom”. In an embodiment, when the one-dimensional feature vector for the i-th atom of the ligand is LFᵢ, the one-dimensional feature vector for the j-th atom of the ligand is LFᵢ, and the two-dimensional feature vector for the i-th atom and the j-th atom is LFᵢⱼ, LFᵢⱼ which is a pair representation related to the i-th atom and the j-th atom may correspond to Linear(concat(LFᵢⱼ, LFᵢ, LFⱼ). In other words, LFᵢⱼ may be embedded based on concatenating LFᵢⱼ, LFᵢ, LFⱼ and applying a linear function to concatenated vectors. Meanwhile, the second pair representation may be generated for each of all atoms pairs, which may be generated by using the atoms included in the ligand (in other words, when the ligand includes m atoms, m×m second pair representations may be generated).

Step S320 above is a step of obtaining an interaction representation between the protein and the ligand.

In relation to step S320 above, the processor 110 may perform “an operation of obtaining the interaction representation between the protein and the ligand by using a one-dimensional feature vector calculated based on one-dimensional information of the protein and a one-dimensional feature vector calculated based on one-dimensional information of the ligand”. At this time, “the one-dimensional feature vector calculated based on the one-dimensional information of the protein” may correspond to “the one-dimensional feature vector generated based on the feature (e.g., the structural feature, the physical/chemical property feature, etc.) of individual amino acid residues of the protein” as described above. Further, “the one-dimensional feature vector calculated based on the one-dimensional information of the ligand” may correspond to “the one-dimensional feature vector generated based on the feature (e.g., the structural feature, the physical/chemical property feature, etc.) of individual atoms of the ligand” as described above.

Specifically, in relation to step S320 above, the processor 110 may perform “an operation of obtaining an interaction representation related to the i-th atom of the ligand and the j-th amino acid residue of the protein by using the one-dimensional feature vector for the i-th atom of the ligand and the one-dimensional feature vector for the j-th amino acid residue of the protein. For example, when the one-dimensional feature vector for the i-th atom of the ligand is LFᵢ and the one-dimensional feature vector for the j-th residue of the protein is PFⱼ, Iᵢⱼ which is the interaction representation related to the i-th atom of the ligand and the j-th amino acid residue of the protein may correspond to Linear(concat(LFᵢ, PFⱼ)). In other words, Iᵢⱼ may be embedded based on concatenating LFᵢ, PFⱼ and applying a linear function to concatenated vectors. Meanwhile, the interaction representation may be generated for each of all pairs or combinations of “amino acid residues and atoms”, which may be generated by using the amino acid residues included in the protein and the atoms included in the ligand (in other words, when the protein includes n amino acid residues and the ligand includes m atoms, m×n interaction representations may be generated).

Step S330 above is a step of updating the first pair representation, the second pair representation, and the interaction representation.

In relation to step S330 above, the processor 110 may perform “a step of updating the first pair representation, the second pair representation, and the interaction representation by using a first neural network model” (S331). In this case, the first neural network model may include a first-first neural network model that independently updates each of the first pair representation, the second pair representation, and the interaction representation, a first-second neural network model that updates at least one of the first pair representation or the second pair representation by using the interaction representation; and a first-third neural network model that updates the interaction representation by using at least one of the first pair representation or the second pair representation.

In an embodiment, the first-first neural network model may include a neural network model that independently updates each of the first pair representation, the second pair representation, and the interaction representation by using a self-attention mechanism. Furthermore, the first-second neural network model may include \textcircled{1} a neural network model that updates a pair representation associated with the i-th amino acid residue of the protein and the j-th amino acid residue of the protein, based on an interaction representation associated with the i-th amino acid residue of the protein and an interaction representation associated with the j-th amino acid residue of the protein. Specifically, when updating a first pair representation Pᵢⱼ corresponding to the i-th and j-th protein residues, the first-second neural network model may use “an interaction representation associated with the i-th amino acid residue of the protein (e.g., when a matrix (m×n matrix) of interaction representations between the protein and the ligand is implemented in a form having rows corresponding to atoms of the ligand and columns corresponding to amino acid residues of the protein, columns (I₁ᵢ, I₂ᵢ, . . . Iₘᵢ) of the interaction representation corresponding to the i-th amino acid residue in the matrix of the interaction representations)” and “an interaction representation associated with the j-th amino acid residue of the protein (columns (I₁ᵢ, I₂ᵢ, . . . Iₘᵢ) of the interaction representation corresponding to the j-th amino acid residue in the matrix of the interaction representations)”. For example, the first-second neural network model may perform linear transform or pooling for each of “the column (I₁ᵢ, I₂ᵢ, . . . Iₘᵢ) of the interaction representation corresponding to the i-th amino acid residue” and “the column (I₁ⱼ, I₂ⱼ, . . . Iₘⱼ) of the interaction representation corresponding to the j-th amino acid residue”, and then update the Pᵢⱼ based on an average value for rows of an element-wise product of the two. Furthermore, the first-second neural network model may include \textcircled{2} a neural network model that updates a pair representation associated with the i-th atom of the ligand and the j-th atom of the ligand, based on an interaction representation associated with the i-th atom of the ligand and the interaction representation associated with the j-th atom of the ligand. Specifically, when updating a second pair representation Lᵢⱼ corresponding to the i-th and j-th atoms, the first-second neural network model may use “an interaction representation associated with the i-th atom of the ligand (e.g., when a matrix (m×n matrix) of interaction representations between the protein and the ligand is implemented in a form having rows corresponding to atoms of the ligand and columns corresponding to amino acid residues of the protein, rows (Iᵢ₁, I₂ⱼ, . . . Iᵢₙ) of the interaction representation corresponding to the i-th atom in the matrix of the interaction representations)” and “an interaction representation associated with the j-th atom of the ligand (rows (Iⱼ₁, Iⱼ₂, . . . Iⱼₙ) of the interaction representation corresponding to the j-th atom in the matrix of the interaction representations)”. For example, the first-second neural network model may perform linear transform or pooling for each of “the row (Iᵢ₁, Iᵢ₂, . . . Iᵢₙ) of the interaction representation corresponding to the i-th atom” and “the row (Iⱼ₁, Iⱼ₂, . . . Iⱼₙ) of the interaction representation corresponding to the j-th atom”, and then update the Lᵢⱼ based on an average value for columns of an element-wise product of the two.

Further, the first-third neural network model may include \textcircled{1} a neural network model that updates the interaction representation related to the i-th atom of the ligand by using a cross-attention mechanism that refers to the first pair representation related to the protein. Specifically, when updating “an interaction representation related to the i-th atom of the ligand (e.g., when a matrix (m×n matrix) of interaction representations between the protein and the ligand is implemented in a form having rows corresponding to atoms of the ligand and columns corresponding to amino acid residues of the protein, the row of the interaction representation corresponding to the i-th atom in the matrix of interaction representations)”, the first-third neural network model may use an attention mechanism between interaction representations related to the i-th atom of the ligand (e.g., an attention mechanism implementing message passing between interaction representations, where 1≤k≤n, 1≤p≤n), and in particular, may utilize a cross-attention mechanism that refers to the first pair representation related to the protein during the process of such attention mechanism (e.g., referring to Pₖₚ for a k-th amino acid and a p-th amino acid associated with message passing in the process of message passing from Iᵢₖ to Iᵢₚ). In an embodiment, the cross-attention mechanism referring to the first pair representation may include an attention mechanism wherein at least one of Query (Q), Key (K), or Value (V) value is computed based on the first pair representation. Further, the first-third neural network model may include \textcircled{2} a neural network model that updates the interaction representation related to the j-th amino acid residue of the protein by using the cross-attention mechanism that refers to the second pair representation related to the ligand. Specifically, when updating “the interaction representation related to the j-th amino acid residue of the protein (e.g., when a matrix (m×n matrix) of interaction representations between the protein and the ligand is implemented in a form having rows corresponding to atoms of the ligand and columns corresponding to amino acid residues of the protein, a column (I₁ⱼ, I₂ⱼ, . . . Iₘⱼ) of the interaction representation related to the j-th amino acid residue of the protein)”, the first-third neural network model may use an attention mechanism between interaction representations related to the j-th amino acid residue of the protein (e.g., an attention mechanism implementing message passing between I_{qj} and Iᵣⱼ, where 1≤q≤m, 1≤r≤m), and in particular, may utilize a cross-attention mechanism that refers to the second pair representation related to the ligand during the process of such attention mechanism (e.g., referring to L_{qr} for a q-th atom and an r-th atom associated with message passing in the process of message passing from I_{qj} to Iᵣⱼ). In an embodiment, the cross-attention mechanism referring to the second pair representation may include an attention mechanism wherein at least one of Query (Q), Key (K), or Value (V) value is computed based on the second pair representation.

The first-second neural network model and the first-third neural network model may implement a process in which information is exchanged between “first pair representations related to the protein”, “second pair representations related to the ligand”, and “interaction representations between the protein and the ligand”. For example, when updating the first pair representations or the second pair representations, the first-second neural network model may use the interaction representations together, and when updating the interaction representations, the first-third neural network model may use the first pair representations or the second pair representations together. Therefore, through such mutual organic update processes, the first pair representations, the second pair representations, and the interaction representations may be updated to better reflect structural changes caused by binding between the protein and the ligand.

Meanwhile, in relation to step S330 above, the processor 110 may update a plurality of first pair representations related to the protein or a plurality of second pair representations related to the ligand to have symmetry. Specifically, the processor 110 may, in an intermediate step or a final step of the updating process (e.g., a final step before being input to a second neural network model to be described below), force Pᵢⱼ and Pⱼᵢ included in the plurality of first pair representations to have identical values to each other, or force Lᵢⱼ and Lⱼᵢ included in the plurality of second pair representations to have identical values to each other. Therefore, through such processes, substantially identical pair representations may be forced to influence a prediction process with identical values to each other, and as a result, the accuracy of binding structure prediction may be further enhanced.

In an embodiment, the processor 110 may perform “a step of updating a first matrix including the plurality of first pair representations related to the protein” (S332) and “a step of updating a second matrix including the plurality of second pair representations associated with the ligand” (S333). For example, when the protein includes n amino acid residues, the processor 110 may update a first matrix (matrix P of n×n) consisting of first pair representations for n×n amino acid pairs, and when the ligand includes m atoms, the processor 110 may update a second matrix (matrix L of m×m) consisting of second pair representations for m×m atom pairs. Further, the processor 110 may perform update so that at least one of the first matrix or the second matrix becomes a symmetric matrix. For example, the processor 110 may force components of the first matrix to satisfy a condition of Pᵢⱼ=Pⱼᵢ (1≤i≤n, 1≤j≤n) in order to force the first matrix to become the symmetric matrix. In other words, the processor 110 may force Pᵢⱼ and Pⱼᵢ to have identical values to each other, and in this case, the forced identical value may be variously configured as an average value, a maximum value, a minimum value, or the like. Further, the processor 110 may force components of the second matrix to satisfy a condition of Lᵢⱼ=Lⱼᵢ (1≤i≤m, 1≤j≤m) in order to force the second matrix to become the symmetric matrix. In other words, the processor 110 may force Lᵢⱼ and Lⱼᵢ to have identical values to each other, and in this case, the forced identical value may be variously configured as an average value, a maximum value, a minimum value, or the like.

Step S340 above is a step of predicting a binding structure between the protein and the ligand after the updating, based on the first pair representation, the second pair representation, and the interaction representation.

In relation to step S340 above, the processor 110 may perform “a step of predicting the binding structure between the protein and the ligand using a second neural network model” (S341).

In the present disclosure, the “binding structure” may include information regarding a three-dimensional structure of the protein and the ligand output from a final layer of the second neural network model, and may include information related to coordinates of atoms constituting a binding site when the protein and the ligand bind, or information related to interactions between respective amino acid residues included in the protein and respective atoms included in the ligand.

Further, in the present disclosure, the binding structure may include information modeled by a scoring function that is shown to adequately satisfy a prediction value after comparing scoring functions created based on predicted values such as distances between amino acid residues included in the protein, distances between atoms included in in the ligand, and distances between the protein and the ligand with existing physics-based scoring functions.

Further, the second neural network model may include: a second-first neural network model that calculates a protein representation using the first pair representation and the interaction representation, a second-second neural network model that calculates a ligand representation using the second pair representation and the interaction representation, and a second-third neural network model that calculates the binding structure between the protein and the ligand using the protein representation, the ligand representation, and the interaction representation. In an embodiment, the processor 110 may output a “one-dimensional protein representation (e.g., a representation associated with individual amino acids of the protein)” using the [“first pair representation”, “second pair representation”, “interaction representation”] based on the second-first neural network model, output a “one-dimensional ligand representation (e.g., a representation associated with individual atoms of the ligand)” using the [“first pair representation”, “second pair representation”, “interaction representation”] based on the second-second neural network model, and predict the “binding structure of the protein and ligand (e.g., coordinates of amino acid residues of the protein and atoms of the ligand)” using the [“one-dimensional protein representation”, “one-dimensional ligand representation”, “interaction representation”] based on the second-third neural network model.

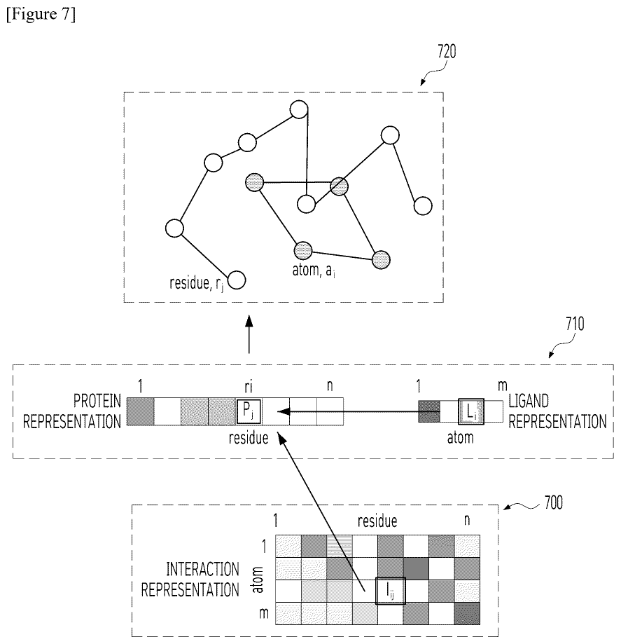

In this case, the second-third neural network model may perform a message passing process between the protein representation and the ligand representation, and in particular, may perform an operation that refers to the interaction representation during the message passing process. Therefore, through such an operation of the second-third neural network model, structural changes caused by binding may be more readily reflected to a prediction result. Meanwhile, the message passing process may be implemented based on the cross-attention mechanism or the graph neural network (GNN). In an embodiment, the second-third neural network model may, as illustrated in FIG. 7, implement message passing from Lᵢ (1≤i≤m), which is the one-dimensional ligand representation of the i-th atom of the ligand to Pᵢ (1≤j≤n), which is the one the one-dimensional protein representation of the j-th amino acid residue of the protein, and in particular, may perform an operation that refers to Iᵢⱼ, which is an interaction representation of the i-th atom of the ligand and the j-th amino acid residue of the protein during the message passing process. Meanwhile, when the message passing from the Lᵢ to the Pⱼ is implemented by the cross-attention mechanism, a cross-attention mechanism that refers to the Iᵢⱼ may include an attention mechanism in which at least one of Query (Q), Key (K), or Value (V) value is computed additionally based on the Iᵢⱼ. Alternatively, when the message passing from the Lᵢ to the Pⱼ is implemented based on the graph neural network (GNN), the Lᵢ and the Pⱼ may be composed of nodes of the graph, and edge information or message passing between the nodes may be computed additionally based on the Iᵢⱼ.

As another embodiment related to step S340 above, the processor 110 may, instead of directly predicting the binding structure based on the representations (i.e., the first pair representation, the second pair representation, and the interaction representation), perform operations of computing a scoring functions based on predicted values based on the representations, and then modifying the binding structure based on computed scores. For example, the processor 110 may perform “a step of calculate distance information related to the protein or the ligand based on the first pair representation, the second pair representation, and the interaction representation by using the third neural network model” (S342), and “a step of predicting the binding structure between the protein and the ligand by using a first scoring function based on the distance information and a second scoring function based on physics” (S343). In an embodiment, the third neural network model may adopt a model that predicts “inter-residue distances of the protein, inter-atomic distances of the ligand, and mutual distances in the protein-ligand binding structure” to create a scoring function (i.e., the first scoring function), and then mixes the created scoring function with existing physics based scoring functions (i.e., the second scoring function) to predict a value that satisfies the mixed scoring function (i.e., a model that predicts a value that satisfies both the first scoring function and the second scoring function).

According to an embodiment of the present disclosure, the processor 110 may further perform, following steps S300 to S340 mentioned above, “a step of re-obtaining the first pair representation, the second pair representation, and the interaction representation based on the predicted binding structure” (S350), “a step of updating the first pair representation, the second pair representation, and the interaction representation” (S360), and “a step of re-predicting the binding structure between the protein and the ligand after the updating, based on the first pair representation, the second pair representation, and the interaction representation” (S370). In this case, steps S350 to S370 above may correspond to repeating steps S300 to S340 above repeated by treating the predicted binding structure as a new input structure. Therefore, step S350 above may include operations corresponding to steps S300, S310, and S320 mentioned above, step S360 above may include operations corresponding to step S330 above, and step S370 above may include operations corresponding to step S340 above. Meanwhile, the processor 110 may perform such iterative repeated predictions multiple times, and through such iterative repeated predictions, iterative refinement of the binding structure may be implemented. For example, the processor 110 may perform such iterative repeated predictions N times (e.g., perform a second prediction based on a first prediction structure, perform a third prediction based on a second prediction structure, and perform an N-th prediction based on an (N-1)-th prediction structure) to output a final N-th prediction result. Through such iterative repeated predictions, the protein-ligand binding structure is continuously improved based on a binding structure of a previous time, thereby further enhancing prediction accuracy of the binding structure.

According to an embodiment of the present disclosure, the processor 110 may perform a training operation of training the neural network model. For example, the processor 110 may additionally perform “a step calculating a loss function for training the neural network model”. Such a training operation may be implemented in association with steps S300 to S370 above performed based on training data. Further, the training operation may include an operation of training at least one of the first neural network model, the second neural network model, or the third neural network model.

In an embodiment, the processor 110 may use a loss function related to protein information on the predicted protein-ligand binding structure. For example, the processor 110 may use a loss function calculated based on inter-protein residue distances and angles, Frame Aligned Point Error (FAPE), stereo chemistry information, and the like. Further, the loss function may be related to a root mean square deviation (RMSD) based on physical differences between the predicted protein structure and ground truth data.

Further, the processor 110 may use a loss function related to ligand information on the predicted protein-ligand binding structure. For example, the processor 110 may use a loss function calculated based on inter-atom distances and a torsion angle of the ligand, stereo chemistry information, and the like. Further, the loss function may be related to a root mean square deviation based on physical differences between the predicted ligand structure and ground truth data.

Further, the processor 110 may use a loss function related to protein-ligand interaction information on the predicted protein-ligand binding structure. For example, the processor may utilize a loss function calculated based on a distance between a protein residue and a ligand atom, binding types (e.g., H-bond, pi-interaction, salt bridge, etc.), protein-ligand binding force, Frame Aligned Point Error (FAPE) for the protein-ligand binding structure, and the like. Additionally, the processor 110 may directly train a protein-ligand interaction by using the loss function related to the interaction information as an auxiliary loss function. For example, “a step of calculating the auxiliary loss function based on the interaction information between the protein and the ligand” may be additionally performed. By training the neural network model through such an additional auxiliary loss function, the neural network model may be made to better predict the interaction. Further, the aforementioned interaction representations may also be extracted more accurately.

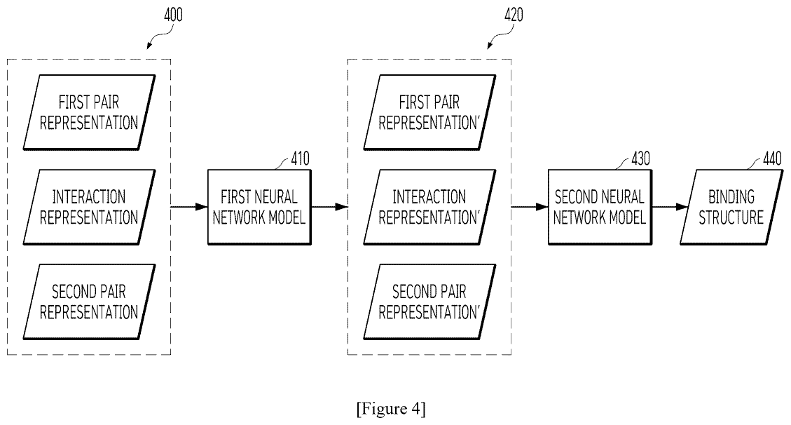

Hereinafter, referring to FIG. 4, an embodiment of a method for predicting a protein-ligand binding structure using a neural network model is disclosed.

Referring to FIG. 4, the processor 110 may use a first neural network model, a second neural network model, and a third neural network model in order to predict a binding structure 440 for a protein and a ligand. The processor 110 may generate “updated first pair representation', second pair representation', and interaction representation 420” by using the first neural network model 410 based on “first pair representation related to the protein, second pair representation related to the ligand, and interaction representation related to an interaction between the protein and the ligand 400”. In this case, the processor may perform an operation of independently updating each of the first pair representation', the second pair representation', and the interaction representation, based on the first-first neural network model of the first neural network model 410. Further, the processor 110 may perform an operation of additionally considering the interaction representation when updating at least one of the first pair representation or the second pair representation, based on the first-second neural network model of the first neural network model 410. In this case, the processor 110 may perform an operation of additionally considering at least one of the first pair representation or the second pair representation when updating the interaction representation based on the first-third neural network model of the first neural network model 410.

Next, the processor 110 may predict the binding structure 440 of the protein and ligand using a second neural network model 430 based on the updated first pair representation, second pair representation, and interaction representation 420. In an embodiment, the processor 110 may generate a protein representation (e.g., a one-dimensional protein representation) based on the updated first pair representation and the updated interaction representation by using the second-first neural network model of the second neural network model 430. Further, the processor 110 may generate a ligand representation (e.g., a one-dimensional protein representation) based on the updated second pair representation and the updated interaction representation by using the second-second neural network model of the second neural network model 430. Furthermore, the processor 110 may predict the binding structure between the protein and the ligand based on the protein representation, the ligand representation, and the interaction representation by using the second-third neural network model of the second neural network model 430.

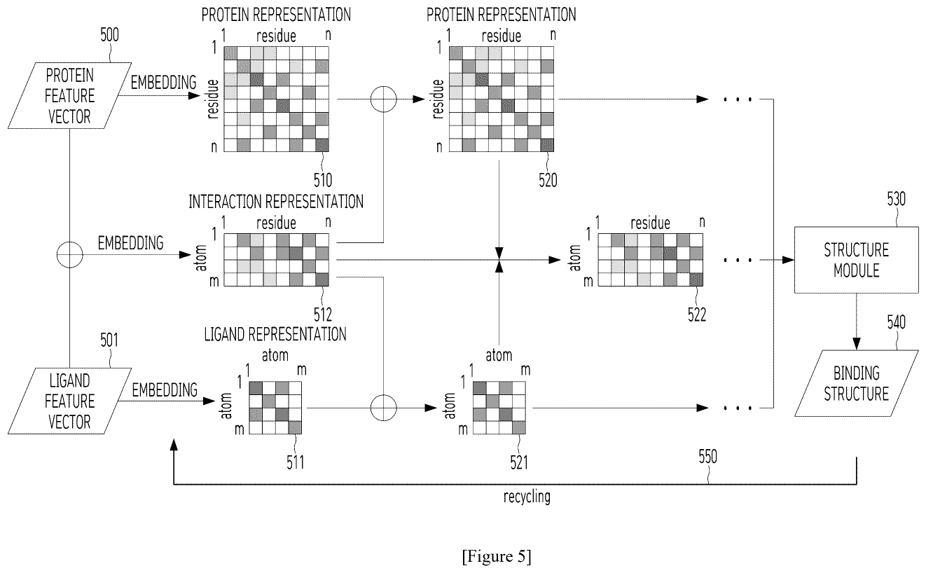

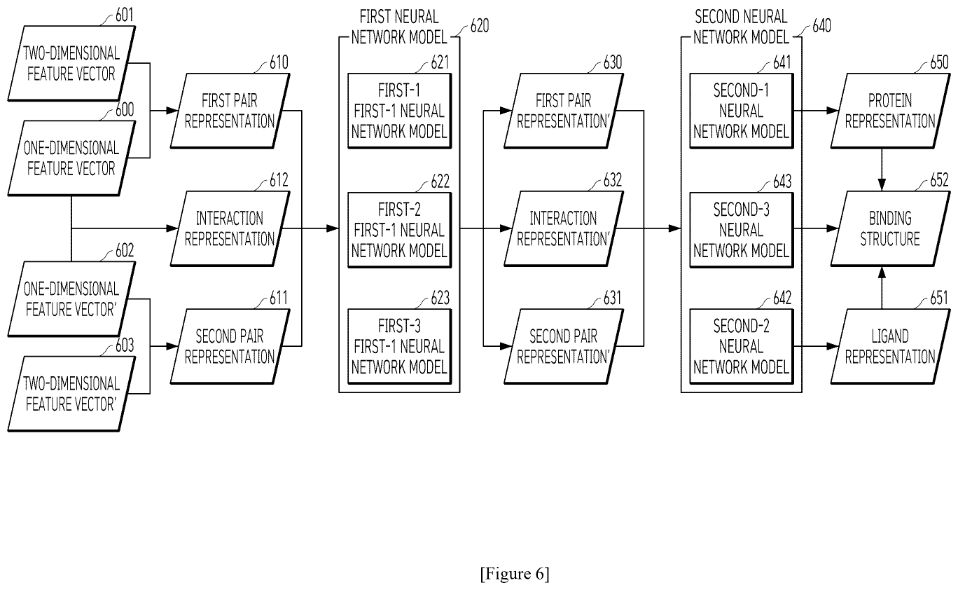

Meanwhile, alternatively, the processor 110 may predict the binding structure 440 of the protein and ligand using a third neural network model (not illustrated) based on the updated first pair representation, second pair representation, and interaction representation 420”. Specifically, the processor 110 may perform “an operation of calculating distance information related to the protein or the ligand based on the updated first pair representation, the updated second pair representation, and the updated interaction by using the third neural network model”, and “an operation of predicting the binding structure between the protein and the ligand by using the first scoring function based on the distance information and the second scoring function based on the physics”. Hereinafter, referring to FIGS. 5 to 7, a more specific embodiment of a method for predicting a protein-ligand binding structure using a neural network model is disclosed. First, referring to FIGS. 5 and 6, the processor 110 may extract feature vectors 500 related to a structure or a property of the protein. For example, the processor 110 may calculate a one-dimensional feature vector (PFᵢ, PFⱼ) 600 based on one-dimensional information related to the structure or property of the protein, and calculate a two-dimensional feature vector (PFᵢⱼ) 601 based on two-dimensional information related to the structure or property of the protein. Further, the processor 110 may calculated feature vectors 501 related to a structure or a property of the ligand. For example, the processor 110 may calculate a one-dimensional feature vector (LFᵢ, LFⱼ) 602 based on one-dimensional information related to the structure or property of the ligand, and calculate a two-dimensional feature vector (LFᵢⱼ) 603 based on two-dimensional information related to the structure or property of the protein as described above.

Next, the processor 110 may obtain the first pair representations 510 and 610 related to the protein through an embedding process using one-dimensional feature vectors (PFᵢ and PFⱼ) 600 for the protein and a two-dimensional feature vector (PFᵢⱼ) 601 for the protein. Here, when the protein includes n amino acid residues, the first pair representations 510 and 610 may be implemented in a form of an n×n matrix. Further, the processor 110 may obtain second pair representations (Lᵢⱼ) 511 and 611 related to the ligand through an embedding process using one-dimensional feature vectors (LFᵢ, LFⱼ) 602 for the ligand and a two-dimensional feature vector (LFᵢⱼ) 603 for the ligand. Here, when the ligand includes m atoms, the second pair representations (Lᵢⱼ) 511 and 611 may be implemented in a form of an m×m matrix. Further, the processor 110 may obtain the interaction representations (Iᵢⱼ) 512 and 612 through an embedding process using one-dimensional feature vectors (LFᵢ, LFⱼ) 602 for the ligand and one-dimensional feature vectors (PFᵢ, PFⱼ) 600 for the protein. Here, when the ligand includes m atoms and the protein includes n amino acid residues, the interaction representations (Iᵢⱼ) 512 and 612 may be implemented in a form of m×n.

Next, the processor 110 may generate updated first pair representations (P′ᵢⱼ) 520 and 630, updated second pair representations (L′ᵢⱼ) 521 and 631, and updated interaction representations (I′ᵢⱼ) 522 and 632 by using the first neural network model 620 based on the first pair representations (Pᵢⱼ) 510 and 610, the second pair representations (Lᵢⱼ) 511 and 611, and the interaction representations (Iᵢⱼ) 512 and 612. Specifically, the processor 110 may generate the updated first pair representations (P′ᵢⱼ) 520 and 630 by using a first-first neural network model 621 based on the first pair representations (Pᵢⱼ) 510 and 610 and a first-second neural network model 622 based on the first pair representations (P′ᵢⱼ) 510 and 610 and the interaction representations (Iᵢⱼ) 512 and 612. At this time, when the protein includes n amino acid residues, the updated first pair representations (P′ᵢⱼ) 520 and 630 may be implemented in the form of the n×n matrix similarly as before the updating. Further, the updated first pair representations (P′ᵢⱼ) 520 and 630 may be updated while being forced to have symmetry. In addition, the processor 110 may generate updated second pair representations (L′ᵢⱼ) 521 and 631 by using the first-first neural network model 621 based on the second pair representations (Lᵢⱼ) 511 and 611 and the first-second neural network model 622 based on the second pair representations (Lⱼᵢ) 511 and 611 and the interaction representations (Iᵢⱼ) 512 and 612. At this time, when the ligand includes m atoms, the updated second pair representations (L′ᵢⱼ) 521 and 631 may be implemented in the form of the m×m matrix similarly as before the updating. Further, the updated second pair representations (L′ᵢⱼ) 521 and 631 may be updated while being forced to have symmetry (Lᵢⱼ=Lⱼᵢ). Further, the processor 110 may generate updated interaction representations (I′ᵢⱼ) 522 and 632 by using the first-third neural network model 622 based on the first pair representations (Pᵢⱼ) 510 and 610, the second pair representations (Lᵢⱼ) 511 and 611, and the interaction representations (Iᵢⱼ) 512 and 612. Here, when the ligand includes m atoms and the protein includes n amino acid residues, the interaction representations (I′ᵢⱼ) 522 and 632 may be implemented in the form of m×n similarly as before the updating. Further, as described above, the processor 110 may use a cross-attention mechanism that refers to the first pair representations (Pᵢⱼ) 510 and 610 related to the protein (for example, refers to Pₖₚ for a k-th amino acid and a p-th amino acid associated with message passing during a message passing process from Iᵢₖ to Iᵢₚ) when updating rows (Iᵢ₁, Iᵢ₂, . . . , Iᵢₙ) of interaction representations corresponding to the i-th atom of the ligand in the matrix of the interaction representations (Iᵢⱼ) 512 and 612. Further, as described above, the processor 110 may use a cross-attention mechanism that refers to the second pair representations (Iᵢⱼ) 511 and 611 related to the ligand (for example, refers to L_{qr} for a q-th atom and a r-th atom associated with message passing during a message passing process from I_{qi} to Iᵣᵢ) when updating columns (I₁ⱼ, I₂ⱼ, . . . , Iₘⱼ) of interaction representations corresponding to the j-th amino acid residues of the protein in the matrix of the interaction representations (Iᵢⱼ) 512 and 612.

Next, the processor 110 may generate binding structures 540 and 652 by using the second neural network models (structure module) 530 and 640 based on the updated first pair representations (P′ᵢⱼ) 520 and 630, the updated second pair representations (L′ᵢⱼ) 521 and 631, and the updated interaction representations (I′ᵢⱼ) 521 and 632. Specifically, the processor 110 may output a ‘one-dimensional protein representation 650 (for example, representations associated with individual amino acids of the protein)’ by using based on the updated first pair representations (P′ᵢⱼ) 520 and 630, the updated second pair representations (L′ᵢⱼ) 521 and 631, and the updated interaction representations (I′ᵢⱼ) 522 and 632 based on a second-first neural network model 641 included in the second neural network models (structure module) 530 and 640. Further, the processor 110 may output a ‘one-dimensional ligand representation 651 (for example, representations associated with individual atoms of the ligand)’ by using based on the updated first pair representations (P′ᵢⱼ) 520 and 630, the updated second pair representations (L′ᵢⱼ) 521 and 631, and the updated interaction representations (I′ᵢⱼ) 52 and 632 based on a second-second neural network model 642 included in the second neural network models (structure module) 530 and 640. Furthermore, the processor 110 may, based on a second-third neural network model 643 included in the second neural network models (structure module) 530 and 640, predict protein-ligand binding structures 540 and 652 (for example, coordinates of the amino acid residue of the protein and the atoms of the ligand)′ by using the one-dimensional protein representation 650, the one-dimensional ligand representation 651, and the updated interaction representations (I′ᵢⱼ) 522 and 632. In this case, the second-third neural network model 643 may perform a message passing process between the one-dimensional protein representation 650 and the one-dimensional ligand representation 651, and in particular, may perform an operation that refers to the updated interaction representations (I′ᵢⱼ) 522 and 632 during the message passing process. Therefore, through such an operation of the second-third neural network model 643, structural changes caused by binding may be more readily reflected to a prediction result. Meanwhile, the message passing process may be implemented based on the cross-attention mechanism or the graph neural network (GNN).

Referring to FIG. 7 to further examine the second-third neural network model 643, the second-third neural network model 643 may, as illustrated in FIG. 7, implement message passing from Lᵢ (1≤i≤m) which is the one-dimensional ligand representation 651 of the i-th atom of the ligand to Pⱼ (1≤j≤n) which is the one-dimensional protein representation 650 of the j-th amino acid residue of the protein. In particular, during such a message passing process, operations may be performed that refers to the updated interaction representations (Iᵢⱼ) 522 and 632 which are interaction representations of the i-th atom of the ligand and the j-th amino acid residue of the protein. Meanwhile, when the message passing from the Lᵢ 651 to the Pⱼ 650 is implemented by the cross-attention mechanism, a cross-attention mechanism that refers to the updated Iᵢⱼ 522 and 632 may include an attention mechanism in which at least one of Query (Q), Key (K), or Value (V) value is computed additionally based on the updated Iᵢⱼ 522 and 632. Alternatively, when the message passing from the Lᵢ to Pⱼ the implemented based on the graph neural network (GNN), the Lᵢ 651 and the Pⱼ 650 may be composed of nodes of the graph, and edge information or message passing between the nodes may be computed additionally based on the Iᵢⱼ 522 and 632.

Meanwhile, the processor 110 may, as mentioned above, re-obtain the first pair representations 510 and 610, the second pair representations 511 and 611, and the interaction representations 512 and 612 based on the binding structures 540 and 652 of the protein and the ligand predicted by the second neural network models (structure module) 530 and 640, and perform operations of repeat subsequent processes. For example, the processor 110 may, based on the predicted binding structures 540 and 652 of the protein and the ligand, re-calculate the protein feature vector and the ligand feature vector, and may re-obtain the first pair representations 510 and 610, the second pair representations 511 and 611, and the interaction representation 512 and 612 based on the calculated feature vectors. Further, the processor 110 may perform “operations to update the first pair representations 510 and 610, the second pair representations 511 and 611, and the interaction representations 512 and 612” and “operations of re-predicting the binding structure between the protein and the ligand based on the updated representations”. In other words, the processor 110 may perform a process, in which the predicted binding structure between the protein and the ligand passes through the neural network model again, one or more times to obtain a repeatedly refined final binding structure.

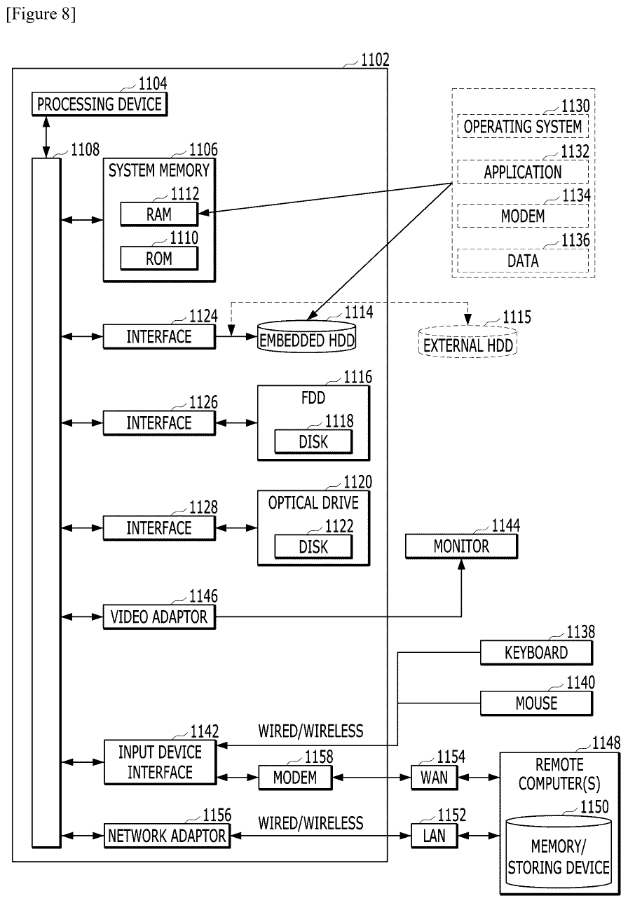

FIG. 8 is a simple and normal schematic view of an exemplary computing environment in which the exemplary embodiments of the present disclosure may be implemented.

The present disclosure has been described as being generally implementable by the computing device, but those skilled in the art will appreciate well that the present disclosure is combined with computer executable commands and/or other program modules executable in one or more computers and/or be implemented by a combination of hardware and software.

In general, a program module includes a routine, a program, a component, a data structure, and the like performing a specific task or implementing a specific abstract data form. Further, those skilled in the art will well appreciate that the method of the present disclosure may be carried out by a personal computer, a hand-held computing device, a microprocessor-based or programmable home appliance (each of which may be connected with one or more relevant devices and be operated), and other computer system configurations, as well as a single-processor or multiprocessor computer system, a mini computer, and a main frame computer.

The embodiments of the present disclosure may be carried out in a distribution computing environment, in which certain tasks are performed by remote processing devices connected through a communication network. In the distribution computing environment, a program module may be located in both a local memory storage device and a remote memory storage device.

The computer generally includes various computer readable media. The computer accessible medium may be any type of computer readable medium, and the computer readable medium includes volatile and non-volatile media, transitory and non-transitory media, and portable and non-portable media. As a non-limited example, the computer readable medium may include a computer readable storage medium and a computer readable transport medium. The computer readable storage medium includes volatile and non-volatile media, transitory and non-transitory media, and portable and non-portable media constructed by a predetermined method or technology, which stores information, such as a computer readable command, a data structure, a program module, or other data. The computer readable storage medium includes a RAM, a Read Only Memory (ROM), an Electrically Erasable and Programmable ROM (EEPROM), a flash memory, or other memory technologies, a Compact Disc (CD)-ROM, a Digital Video Disk (DVD), or other optical disk storage devices, a magnetic cassette, a magnetic tape, a magnetic disk storage device, or other magnetic storage device, or other predetermined media, which are accessible by a computer and are used for storing desired information, but is not limited thereto.

The computer readable transport medium generally implements a computer readable command, a data structure, a program module, or other data in a modulated data signal, such as a carrier wave or other transport mechanisms, and includes all of the information transport media. The modulated data signal means a signal, of which one or more of the characteristics are set or changed so as to encode information within the signal. As a non-limited example, the computer readable transport medium includes a wired medium, such as a wired network or a direct-wired connection, and a wireless medium, such as sound, Radio Frequency (RF), infrared rays, and other wireless media. A combination of the predetermined media among the foregoing media is also included in a range of the computer readable transport medium.

An illustrative environment 1100 including a computer 1102 and implementing several aspects of the present disclosure is illustrated, and the computer 1102 includes a processing device 1104, a system memory 1106, and a system bus 1108. The system bus 1108 connects system components including the system memory 1106 (not limited) to the processing device 1104. The processing device 1104 may be a predetermined processor among various commonly used processors. A dual processor and other multi-processor architectures may also be used as the processing device 1104.

The system bus 1108 may be a predetermined one among several types of bus structure, which may be additionally connectable to a local bus using a predetermined one among a memory bus, a peripheral device bus, and various common bus architectures. The system memory 1106 includes a ROM 1110, and a RAM 1112. A basic input/output system (BIOS) is stored in a non-volatile memory 1110, such as a ROM, an EPROM, and an EEPROM, and the BIOS includes a basic routing helping a transport of information among the constituent elements within the computer 1102 at a time, such as starting. The RAM 1112 may also include a high-rate RAM, such as a static RAM, for caching data.

The computer 1102 also includes an embedded hard disk drive (HDD) 1114 (for example, enhanced integrated drive electronics (EIDE) and serial advanced technology attachment (SATA))-the embedded HDD 1114 being configured for exterior mounted usage within a proper chassis (not illustrated)-a magnetic floppy disk drive (FDD) 1116 (for example, which is for reading data from a portable diskette 1118 or recording data in the portable diskette 1118), and an optical disk drive 1120 (for example, which is for reading a CD-ROM disk 1122, or reading data from other high-capacity optical media, such as a DVD, or recording data in the high-capacity optical media). A hard disk drive 1114, a magnetic disk drive 1116, and an optical disk drive 1120 may be connected to a system bus 1108 by a hard disk drive interface 1124, a magnetic disk drive interface 1126, and an optical drive interface 1128, respectively. An interface 1124 for implementing an outer mounted drive includes, for example, at least one of or both a universal serial bus (USB) and the Institute of Electrical and Electronics Engineers (IEEE) 1394 interface technology.

The drives and the computer readable media associated with the drives provide non-volatile storage of data, data structures, computer executable commands, and the like. In the case of the computer 1102, the drive and the medium correspond to the storage of random data in an appropriate digital form. In the description of the computer readable media, the HDD, the portable magnetic disk, and the portable optical media, such as a CD, or a DVD, are mentioned, but those skilled in the art will well appreciate that other types of computer readable media, such as a zip drive, a magnetic cassette, a flash memory card, and a cartridge, may also be used in the illustrative operation environment, and the predetermined medium may include computer executable commands for performing the methods of the present disclosure.

A plurality of program modules including an operation system 1130, one or more application programs 1132, other program modules 1134, and program data 1136 may be stored in the drive and the RAM 1112. An entirety or a part of the operation system, the application, the module, and/or data may also be cached in the RAM 1112. It will be well appreciated that the present disclosure may be implemented by several commercially usable operation systems or a combination of operation systems.

A user may input a command and information to the computer 1102 through one or more wired/wireless input devices, for example, a keyboard 1138 and a pointing device, such as a mouse 1140. Other input devices (not illustrated) may be a microphone, an IR remote controller, a joystick, a game pad, a stylus pen, a touch screen, and the like. The foregoing and other input devices are frequently connected to the processing device 1104 through an input device interface 1142 connected to the system bus 1108, but may be connected by other interfaces, such as a parallel port, an IEEE 1394 serial port, a game port, a USB port, an IR interface, and other interfaces.

A monitor 1144 or other types of display devices are also connected to the system bus 1108 through an interface, such as a video adaptor 1146. In addition to the monitor 1144, the computer generally includes other peripheral output devices (not illustrated), such as a speaker and a printer.

The computer 1102 may be operated in a networked environment by using a logical connection to one or more remote computers, such as remote computer(s) 1148, through wired and/or wireless communication. The remote computer(s) 1148 may be a work station, a computing device computer, a router, a personal computer, a portable computer, a microprocessor-based entertainment device, a peer device, and other general network nodes, and generally includes some or an entirety of the constituent elements described for the computer 1102, but only a memory storage device 1150 is illustrated for simplicity. The illustrated logical connection includes a wired/wireless connection to a local area network (LAN) 1152 and/or a larger network, for example, a wide area network (WAN) 1154. The LAN and WAN networking environments are general in an office and a company, and make an enterprise-wide computer network, such as an Intranet, easy, and all of the LAN and WAN networking environments may be connected to a worldwide computer network, for example, the Internet.

When the computer 1102 is used in the LAN networking environment, the computer 1102 is connected to the local network 1152 through a wired and/or wireless communication network interface or an adaptor 1156. The adaptor 1156 may make wired or wireless communication to the LAN 1152 easy, and the LAN 1152 also includes a wireless access point installed therein for the communication with the wireless adaptor 1156. When the computer 1102 is used in the WAN networking environment, the computer 1102 may include a modem 1158, is connected to a communication computing device on a WAN 1154, or includes other means setting communication through the WAN 1154 via the Internet. The modem 1158, which may be an embedded or outer-mounted and wired or wireless device, is connected to the system bus 1108 through a serial port interface 1142. In the networked environment, the program modules described for the computer 1102 or some of the program modules may be stored in a remote memory/storage device 1150. The illustrated network connection is illustrative, and those skilled in the art will appreciate well that other means setting a communication link between the computers may be used.

The computer 1102 performs an operation of communicating with a predetermined wireless device or entity, for example, a printer, a scanner, a desktop and/or portable computer, a portable data assistant (PDA), a communication satellite, predetermined equipment or place related to a wirelessly detectable tag, and a telephone, which is disposed by wireless communication and is operated. The operation includes a wireless fidelity (Wi-Fi) and Bluetooth wireless technology at least. Accordingly, the communication may have a pre-defined structure, such as a network in the related art, or may be simply ad hoc communication between at least two devices.

The Wi-Fi enables a connection to the Internet and the like even without a wire. The Wi-Fi is a wireless technology, such as a cellular phone, which enables the device, for example, the computer, to transmit and receive data indoors and outdoors, that is, in any place within a communication range of a base station. A Wi-Fi network uses a wireless technology, which is called IEEE 802.11 (a, b, g, etc.) for providing a safe, reliable, and high-rate wireless connection. The Wi-Fi may be used for connecting the computer to the computer, the Internet, and the wired network (IEEE 802.3 or Ethernet is used). The Wi-Fi network may be operated at, for example, a data rate of 11 Mbps (802.11a) or 54 Mbps (802.11b) in an unauthorized 2.4 and 5 GHz wireless band, or may be operated in a product including both bands (dual bands).

Those skilled in the art may appreciate that information and signals may be expressed by using predetermined various different technologies and techniques. For example, data, indications, commands, information, signals, bits, symbols, and chips referable in the foregoing description may be expressed with voltages, currents, electromagnetic waves, magnetic fields or particles, optical fields or particles, or a predetermined combination thereof.

Those skilled in the art will appreciate that the various illustrative logical blocks, modules, processors, means, circuits, and algorithm operations described in relationship to the embodiments disclosed herein may be implemented by electronic hardware (for convenience, called “software” herein), various forms of program or design code, or a combination thereof. In order to clearly describe compatibility of the hardware and the software, various illustrative components, blocks, modules, circuits, and operations are generally illustrated above in relation to the functions of the hardware and the software. Whether the function is implemented as hardware or software depends on design limits given to a specific application or an entire system. Those skilled in the art may perform the function described by various schemes for each specific application, but it shall not be construed that the determinations of the performance depart from the scope of the present disclosure.

Various embodiments presented herein may be implemented by a method, a device, or a manufactured article using a standard programming and/or engineering technology. A term “manufactured article” includes a computer program, a carrier, or a medium accessible from a predetermined computer-readable storage device. For example, the computer-readable storage medium includes a magnetic storage device (for example, a hard disk, a floppy disk, and a magnetic strip), an optical disk (for example, a CD and a DVD), a smart card, and a flash memory device (for example, an EEPROM, a card, a stick, and a key drive), but is not limited thereto. Further, various storage media presented herein include one or more devices and/or other machine-readable media for storing information.

It shall be understood that a specific order or a hierarchical structure of the operations included in the presented processes is an example of illustrative accesses. It shall be understood that a specific order or a hierarchical structure of the operations included in the processes may be rearranged within the scope of the present disclosure based on design priorities. The accompanying method claims provide various operations of elements in a sample order, but it does not mean that the claims are limited to the presented specific order or hierarchical structure.

The description of the presented embodiments is provided so as for those skilled in the art to use or carry out the present disclosure. Various modifications of the embodiments may be apparent to those skilled in the art, and general principles defined herein may be applied to other embodiments without departing from the scope of the present disclosure. Accordingly, the present disclosure is not limited to the embodiments suggested herein, and shall be interpreted within the broadest meaning range consistent to the principles and new characteristics presented herein.

### Mode for Invention

Related contents in the best mode for carrying out the present disclosure are described as above.

1. A method for predicting a protein-ligand binding structure using a neural network model, performed by a computing device, the method comprising:
obtaining a first pair representation related to a protein;
obtaining a second pair representation related to a ligand;
obtaining an interaction representation between the protein and the ligand;
updating the first pair representation, the second pair representation, and the interaction representation; and
predicting the protein-ligand binding structure after the updating, based on the first pair representation, the second pair representation, and the interaction representation.

2. The method of claim 1, wherein the obtaining of the first pair representation related to the protein includes:
calculating a one-dimensional feature vector based on one-dimensional information related to a structure or a property of the protein;
calculating a two-dimensional feature vector based on two-dimensional information related to the structure or the property of the protein; and
obtaining the first pair representation based on the one-dimensional feature vector and the two-dimensional feature vector.

3. The method of claim 2, wherein the one-dimensional feature vector includes a feature related to an individual amino acid residue of the protein, and
wherein the two-dimensional feature vector includes features related to two amino acid residues of the protein.

4. The method of claim 3, wherein the obtaining of the first pair representation includes:
obtaining pair representations related to an i-th amino acid residue of the protein and a j-th amino acid residue of the protein based on a one-dimensional feature vector for the i-th amino acid residue, a one-dimensional feature vector for the j-th amino acid residue, and a two-dimensional feature vector for the i-th amino acid residue and the j-th amino acid residue, and
wherein the i and j are natural numbers.

5. The method of claim 1, wherein the obtaining of the second pair representation related to the ligand includes:
calculating a one-dimensional feature vector based on one-dimensional information related to a structure or a property of the ligand;
calculating a two-dimensional feature vector based on two-dimensional information related to the structure or the property of the ligand; and
obtaining the second pair representation based on the one-dimensional feature vector and the two-dimensional feature vector.

6. The method of claim 5, wherein the one-dimensional feature vector includes a feature related to an individual atom of the ligand, and
wherein the two-dimensional feature vector includes features related to two atoms of the ligand.

7. The method of claim 6, wherein the obtaining of the second pair representation includes:
obtaining a pair representation related to an i-th atom of the ligand and a j-th atom of the ligand based on a one-dimensional feature vector for the i-th atom, a one-dimensional feature vector for the j-th atom, and a two-dimensional feature vector for the i-th atom and the j-th atom, and
wherein the i and j are natural numbers.

8. The method of claim 1, wherein the obtaining of the interaction representation between the protein and the ligand includes:
obtaining the interaction representation between the protein and the ligand by using a one-dimensional feature vector calculated based on one-dimensional information of the protein and a one-dimensional feature vector calculated based on one-dimensional information of the ligand.

9. The method of claim 8, wherein the obtaining of the interaction representation between the protein and the ligand includes:
obtaining an interaction representation related to an i-th atom of the ligand and a j-th amino acid residue of the protein by using a one-dimensional feature vector for the i-th atom of the ligand and a one-dimensional feature vector for the j-th amino acid residue of the protein, and
wherein the i and j are natural numbers.

10. The method of claim 1, wherein the updating includes:
updating the first pair representation, the second pair representation, and the interaction representation by using a first neural network model, and
wherein the first neural network model includes:
a first-first neural network model that independently updates each of the first pair representation, the second pair representation, and the interaction representation;
a first-second neural network model that updates at least one of the first pair representation or the second pair representation by using the interaction representation; and
a first-third neural network model that updates the interaction representation by using at least one of the first pair representation or the second pair representation.

11. The method of claim 10, wherein the first-first neural network model includes a neural network model that independently updates each of the first pair representation, the second pair representation, and the interaction representation by using a self-attention mechanism.

12. The method of claim 10, wherein the first-second neural network model includes at least one of:
a neural network model that updates a pair representation associated with an i-th amino acid residue of the protein and a j-th amino acid residue of the protein, based on an interaction representation associated with the i-th amino acid residue of the protein and an interaction representation associated with the j-th amino acid residue of the protein; or
a neural network model that updates a pair representation associated with an i-th atom of the ligand and a j-th atom of the ligand, based on an interaction representation associated with the i-th atom of the ligand and an interaction representation associated with the j-th atom of the ligand, and.
wherein the i and j are natural numbers.

13. The method of claim 10, wherein the first-third neural network model includes at least one of:
a neural network model that updates an interaction representation related to an i-th atom of the ligand by using cross-attention mechanism that refers to the first pair representation related to the protein; or
a neural network model that updates an interaction representation related to a j-th amino acid residue of the protein by using a cross-attention mechanism that refers to the second pair representation related to the ligand, and
wherein the i and j are natural numbers.

14. The method of claim 1, wherein the updating includes at least one of:
updating a first matrix including a plurality of first pair representations related to the protein; or
updating a second matrix including a plurality of second pair representations related to the ligand, and
wherein at least one of the first matrix or the second matrix is updated to be a symmetric matrix.

15. The method of claim 1, wherein the predicting of the binding structure between the protein and the ligand includes:
predicting the binding structure between the protein and the ligand by using a second neural network model, and
wherein the second neural network model includes:
a second-first neural network model that calculates a protein representation by using the first pair representation and the interaction representation;
a second-second neural network model that calculates a ligand representation by using the second pair representation and the interaction representation; and
a second-third neural network model that calculates the binding structure between the protein and the ligand by using the protein representation, the ligand representation, and the interaction representation.

16. (canceled)

17. The method of claim 1, wherein the predicting of the binding structure between the protein and the ligand includes:
calculating distance information related to the protein or the ligand based on the first pair representation, the second pair representation, and the interaction representation by using a third neural network model; and
predicting the binding structure between the protein and the ligand by using a first scoring function based on the distance information and a second scoring function based on physics.

18. The method of claim 1, further comprising:
re-obtaining the first pair representation, the second pair representation, and the interaction representation based on the predicted binding structure;
updating the first pair representation, the second pair representation, and the interaction representation; and
re-predicting the binding structure between the protein and the ligand after the updating, based on the first pair representation, the second pair representation, and the interaction representation.

19. The method of claim 1, further comprising:
calculating a loss function for training the neural network model,
wherein the calculating of the loss function includes calculating an auxiliary loss function based on the interaction information between the protein and the ligand.

20. A device comprising:
at least one processor; and
a memory,
wherein the at least one processor is configured to:
obtain a first pair representation related to a protein;
obtain a second pair representation related to a ligand;
obtain an interaction representation between the protein and the ligand;
update the first pair representation, the second pair representation, and the interaction representation; and
predict a binding structure between the protein and the ligand after the updating, based on the first pair representation, the second pair representation, and the interaction representation.

21. A computer program stored in a non-transitory computer-readable storage medium, wherein when the program is executed by at least one processor, the program allows the at least one processor to perform operations of predicting a protein-ligand binding structure, and the operations comprise:
an operation of obtaining a first pair representation related to a protein;
an operation of obtaining a second pair representation related to a ligand;
an operation of obtaining an interaction representation between the protein and the ligand;
an operation of updating the first pair representation, the second pair representation, and the interaction representation; and
an operation of predicting the binding structure between the protein and the ligand after the updating, based on the first pair representation, the second pair representation, and the interaction representation.
